(19) United States
(12) Patent Application Publication
(10) Pub. No.: US 2022/0304915 A1
(43) Pub. Date: Sep. 29, 2022

(54) Multi-Benefit Personal Care Compositions and Methods for the Same

(71) Applicant: Colgate-Palmolive Company, New York, NY (US)

(72) Inventors: Qiang Wu, Hillsborough, NJ (US); Thomas Boyd, Metuchen, NJ (US)

(73) Assignee: Colgate-Palmolive Company, New York, NY (US)

(21) Appl. No.: 17/250,409

(22) PCT Filed: Nov. 12, 2020
(86) PCT No.: PCT/US2020/070771

§ 371 Date: Jan. 19, 2021

Related U.S. Application Data
(60) Provisional application No. 62/948,512, filed on Dec. 16, 2019.

(51) Int. Cl. A61K 8/92; A61K 8/64; A61Q 19/00

(52) U.S. Cl. CPC A61K 8/922; A61K 8/64; A61Q 19/005; A61Q 19/007

(57) ABSTRACT

Personal care compositions and methods for increasing the amount of antimicrobial peptides produced by skin, reducing inflammation of skin, reducing irritation of skin, reducing reactive oxygen species in skin, or any combination thereof are disclosed. The personal care composition may include a carrier and at least two plant oils. The at least two plant oils may be present in an effective amount to increase the amount of antimicrobial peptides produced by skin, reduce inflammation of skin, reduce irritation of skin, reduce reactive oxygen species in skin, or any combination thereof.

### BACKGROUND

Personal care compositions may often incorporate beneficial components or benefit agents to impart benefits or properties to the compositions. For example, skincare compositions may often incorporate antimicrobial agents to impart antimicrobial properties to the composition. Similarly, skincare compositions may often incorporate antioxidants to impart antiaging properties to the composition. While the incorporation of these benefit agents has exhibited efficacy in enhancing the respective properties of the personal care compositions, the search for new benefit agents exhibiting improved or multiple benefits is ongoing.

What is needed, then, are personal care compositions incorporating improved benefit agents having multiple beneficial properties, and methods for the same.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing a personal care composition including a carrier and at least two plant oils, wherein the two plant oils are present in an effective amount to increase the amount of antimicrobial peptides produced by skin, reduce inflammation of skin, reduce irritation of skin, reduce reactive oxygen species in skin, or a combination thereof.

In at least one example, the at least two plant oils may include flaxseed oil and hemp seed oil.

In at least one example, the flaxseed oil and the hemp seed oil may be present in a ratio of about 0.5:1 to about 4:1, more preferably about 1:1 to about 2:1.

In at least one example, the at least two plant oils may be present in an amount of from greater than 0 weight % to about 40 weight %, based on a total weight of the personal care composition.

In at least one example, at least one of the plant oils is a manufactured oil. In at least one example, at least one of the plant oils is a natural oil.

In at least one example, the personal care composition consists essentially of the carrier and the at least two plant oils.

In at least one example, the personal care composition consists of the carrier and the at least two plant oils.

In at least one example, the carrier is a solid carrier. In another example, the carrier is a liquid carrier.

The foregoing and/or other aspects and utilities embodied in the present disclosure may be achieved by providing a method for preparing any one or more of the personal care compositions disclosed herein. The method may include contacting the carrier and the at least two plant oils with one another.

The foregoing and/or other aspects and utilities embodied in the present disclosure may be achieved by providing a method for treating, preventing, or reducing microbes on skin. The method may include contacting the skin with any one or more of the personal care compositions disclosed herein. The method may further include increasing production of antimicrobial peptides in or on the skin.

In at least one example, contacting the skin increases production of antimicrobial peptide biomarker LL-37 in or on the skin.

In at least one example, contacting the skin increases an innate immune response.

The foregoing and/or other aspects and utilities embodied in the present disclosure may be achieved by providing a method for treating or reducing skin irritation or skin inflammation. The method may include contacting the skin with any one or more of the personal care compositions disclosed herein. The method may further include decreasing the amount of IL-1α in or on the skin.

The foregoing and/or other aspects and utilities embodied in the present disclosure may be achieved by providing a method for treating or reducing reactive oxygen species in or on skin.

The method may include contacting the skin with any one or more of the personal care compositions disclosed herein.

The foregoing and/or other aspects and utilities embodied in the present disclosure may be achieved by providing personal care compositions for use in increasing an innate immune response on skin.

The foregoing and/or other aspects and utilities embodied in the present disclosure may be achieved by providing personal care compositions for use in treating or reducing skin irritation or skin inflammation.

The foregoing and/or other aspects and utilities embodied in the present disclosure may be achieved by providing personal care compositions for use in treating or reducing reactive oxygen species in or on skin.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

The present inventors have surprisingly and unexpectedly discovered that personal care compositions including a synergistic combination of flaxseed oil (FSO) and hemp seed oil (HSO) significantly increased the amount of biomarker LL-37 in skin; and thus, significantly increased the amount of antimicrobial peptides (AMPs) produced by the skin. The surprising and unexpected increase in biomarker LL-37 was demonstrated when FSO and HSO were present in a ratio of less than about 1:2, and the effects were more pronounced in a FSO to HSO ratio of less than or equal to about 1:1.

The present inventors have also surprisingly and unexpectedly discovered that personal care compositions including a synergistic combination of FSO and HSO significantly decreased the measured amount of IL-1α in skin, a marker for skin inflammation and irritation. The surprising and unexpected decrease in IL-1α was demonstrated when FSO and HSO were present in a ratio of greater than about 0.5:1 and less than about 4:1, and the effects were more pronounced in a FSO to HSO ratio of from about 1:1 to about 2:1.

The present inventors have further surprisingly and unexpectedly discovered that personal care compositions including a synergistic combination of FSO and HSO significantly decreased the measured amount of reactive oxygen species (ROS), thereby demonstrating skin protecting benefits against atmospheric or environmental pollution. The surprising and unexpected decrease in ROS was demonstrated when FSO and HSO were present in a ratio of about 1:1.

### COMPOSITIONS

Compositions disclosed herein may be or include a personal care product or a personal care composition thereof. For example, compositions disclosed herein may be a personal care composition, a personal care product, or form a portion of the personal care composition or the personal care product. As used herein, the term or expression “personal care composition” may refer to a composition for topical application to skin of mammals, especially humans. The personal care composition may generally be a leave-on personal care composition or rinse off personal care composition, and may include any product applied to a human body. The personal care composition is preferably a leave-on personal care composition. The personal care composition may be in any suitable form. Illustrative forms of the personal care composition may be or include, but is not limited to, a liquid, a lotion, a cream, a foam, a scrub, a gel, a soap bar, a toner, a substance or composition applied with an implement or via a face mask, or the like. Illustrative personal care compositions may be or include, but are not limited to, cleansers, leave-on skin lotions or creams, emulsion, shampoos, conditioners, shower gels, antiperspirants, deodorants, depilatories, lipsticks, foundations, mascara, sunless tanners, sunscreen lotions, body washes, soaps, including bar soaps and liquid soaps (e.g., liquid hand soaps), face washes, moisturizers, serums, spot treatments, cosmetics, or the like.

In an exemplary implementation, the compositions disclosed herein may be personal care compositions including a carrier and one or more plant oils. In one example, the personal care composition may include a single plant oil. In another example, the personal care composition may include at least two plant oils. In a preferred implementation, the personal care composition includes at least two plant oils in a synergistic amount or ratio. For example, the personal care composition may include a first plant oil and a second plant oil, wherein the first plant oil and the second plant oil are present in a synergistic amount or ratio.

The personal care compositions disclosed herein may be capable of or configured to facilitate, promote, enhance, or otherwise increase the production or formation of AMPs produced by the skin, thereby providing protection on surfaces of the skin against one or more microbes and/or multicellular organisms (e.g., bacteria, viruses, etc.). For example, the personal care composition disclosed herein may be capable of or configured to increase the amount of AMPs produced by the skin when a first plant oil, such as FSO, and a second plant oil, such as HSO, are present in a ratio of greater than about 0.5:1 and less than about 4:1 or in a ratio of from about 1:1 to about 2:1.

The personal care compositions disclosed herein may also be capable of or configured to significantly decrease the measured amount of IL-la, a marker for skin inflammation and irritation. As such, the personal care composition disclosed herein may be capable of or configured to treat, decrease, reduce, or otherwise prevent skin inflammation and/or irritation. For example, the personal care compositions disclosed herein may be capable of or configured to significantly decrease the measured amount of IL-la when a first plant oil, such as FSO, and a second plant oil, such as HSO, are present in a ratio of greater than about 0.5:1 and less than about 4:1, or in a ratio of from about 1:1 to about 2:1. Accordingly, the personal care compositions disclosed herein may be utilized in the treatment and/or prevention of skin irritation or skin inflammation.

The personal care compositions disclosed herein may further be capable of or configured to significantly decreased the measured amount of reactive oxygen species (ROS). As such, the personal care composition disclosed herein may be capable of or configured to treat, decrease, reduce, or prevent damage to the skin and/or provide skin protecting benefits against atmospheric or environmental pollution. The personal care compositions disclosed herein may be capable of or configured to significantly decrease the measured amount of IL-1α when a first plant oil, such as FSO, and a second plant oil, such as HSO, are present in a ratio of about 1:1.

In view of the foregoing, it should be appreciated that the personal care compositions disclosed herein may be capable of or configured to provide multiple benefits to the skin. For example, the personal care compositions disclosed herein may concurrently provide protection on surfaces of the skin against one or more microbes, reduce skin irritation and/or inflammation, and/or provide skin protection benefits against atmospheric or environmental pollution.

The personal care composition may include one or more plant oils. For example, the personal care composition may include a single plant oil or a combination of two or more plant oils. As used herein, “plant oil” may refer to a natural oil that is completely obtained from a plant, or a manufactured oil made by blending at least two components of oil (e.g., triglycerides, saturated and/or unsaturated fatty acids, etc.) to substantially mimic the composition of a natural plant oil or provide an oil substantially similar in composition to a plant oil. For example, a manufactured oil substantially similar in composition to a plant oil may include at least 50 weight %, at least 60 weight %, at least 70 weight %, at least 80 weight %, at least 90 weight %, at least 95 weight %, at least 98 weight %, at least 99 weight %, at least 99.5 weight %, at least 99.9 weight %, or 100 weight % of the components that are naturally found in the plant oil that the manufactured oil is designed to substantially mimic.

Illustrative plant oils may be or include, but are not limited to, palm kernel oil, coconut oil, avocado oil, canola oil, corn oil, cottonseed oil, olive oil, palm oil, high-oleic sunflower oil, mid-oleic sunflower oil, sunflower oil, palm stearin oil, palm kernel olein oil, safflower oil, babassu oil, sweet almond oil, castor oil, canola oil, soybean oil, olive oil, acai oil, andiroba oil, apricot kernel oil, argan oil, passion fruit oil, manila oil, mango oil, shea oil, macadamia nut oil, brazil nut oil, borage oil, copaiba oil, grape seed oil, buriti oil, sesame oil, flaxseed oil or linseed oil, blueberry oil, cranberry oil, blackberry oil, plum oil, raspberry oil, camelina oil, camellia oil, walnut oil, wheat germ oil, calendula oil, cherry kernel oil, cucumber seed oil, papaya oil, aloe vera oil, hemp oil, hemp seed oil, or the like, or mixtures or combinations thereof. In a preferred implementation, the personal care composition includes a synergistic combination of at least two plant oils. For example, the personal care composition includes a synergistic combination of hemp seed oil and flaxseed oil

In at least one implementation, any one or more of the plant oils may be present in the personal care composition in an effective amount or a therapeutically effective amount. As used herein, the expression or term “effective amount of one or more plant oils” or the like may refer to an amount of one or a first plant oil sufficient to interact or work synergistically with another or second plant oil to elicit a response (e.g., biological, medical, etc.) of a tissue, system, animal, or human that is being sought. For example, a first plant oil, such as FSO, may be present in the personal care composition in an effective amount to interact or work synergistically with a second plant oil, such as HSO, to provide protection on surfaces of the skin against one or more microbes, reduce skin irritation and/or inflammation, and/or provide skin protection benefits against atmospheric or environmental pollution.

The amount or concentration of any one or more of the plant oils present in the personal care composition may vary widely. In at least one implementation, any one or more of the plant oils may be present in the personal care composition in an amount sufficient to deliver an effective amount and/or ratio, as disclosed herein, of the one or more plant oils to skin cells when applied to an outer surface of the skin or outer dermis. It should be appreciated that the amount and/or ratio of the one or more plant oils present in the in the personal care composition may be relatively greater than the effective amount, as penetration of the one or more plant oils from the outer dermis to the skin cells may be at least partially determined by varying factors, as is known by those having ordinary skill in the art.

In at least one implementation, the amount of any one or more of the plant oils (e.g., each or a combination) present in the personal care composition may be from greater than 0 weight % to less than or equal to 40 weight %, based on a total weight of the personal care composition. For example, any one or more of the plant oils may be present in the personal care composition in an amount of from greater than 0 weight %, about 0.01 weight %, about 0.1 weight %, about 0.2 weight %, about 0.3 weight %, about 0.4 weight %, about 0.5 weight %, about 0.6 weight %, about 0.7 weight %, about 0.8 weight %, about 0.9 weight %, about 1 weight %, about 1.2 weight %, about 1.4 weight %, about 1.6 weight %, about 1.8 weight %, about 2 weight %, about 3 weight %, about 4 weight %, about 5 weight %, about 6 weight %, about 7 weight %, about 8 weight %, about 9 weight %, about 10 weight %, about 15 weight %, or about 20 weight % to about 25 weight %, about 30 weight %, about 35 weight %, about 40 weight %, based on a total weight of the personal care composition. In another example, any one or more of the plant oils may be present in the personal care composition in an amount of at least 0.00001 wt%, at least 0.0001 weight %, at least 0.001 weight %, at least 0.01 weight %, at least 0.1 weight %, at least 1.0 weight %, at least 1.5 weight %, at least 2.0 weight %, at least 2.5 weight %, at least 3.0 weight %, at least 3.5 weight %, at least 4.0 weight %, at least 5 weight %, at least 10 weight %, at least 20 at least %, at least 30 weight %, or more, based on a total weight of the personal care composition. In yet another example, any one or more of the plant oils may be present in the personal care composition in an amount of from greater than 0 weight % to less than 40 weight %, less than 35 weight %, less than 30 weight %, less than 25 weight %, less than 20 weight %, less than 15 weight %, less than 10 weight %, less than 9 weight %, less than 8 weight %, less than 7 weight %, less than 6 weight %, less than 5 weight %, less than 4 weight %, less than 3 weight %, less than 2 weight %, less than 1 weight %, less than 0.1 weight %, or less than 0.01 weight %, based on a total weight of the personal care composition. In yet another example, any one or more of the plant oils may be present in the personal care composition in an amount of from greater than 0 weight % to about 5 weight %, about 0.01 weight % to about 5 weight %, about 0.1 weight % to about 4.9 weight %, about 0.2 weight % to about 4.8 weight %, about 0.3 weight % to about 4.7 weight %, about 0.4 weight % to about 4.6 weight %, about 0.5 weight % to about 4.5 weight %, about 0.6 weight % to about 4.4 weight %, about 0.7 weight % to about 4.3 weight %, about 0.8 weight % to about 4.2 weight %, about 0.9 weight % to about 4.1 weight %, about 1 weight % to about 4 weight %, about 1.2 weight % to about 3.8 weight %, about 1.4 weight % to about 3.6 weight %, about 1.6 weight % to about 3.4 weight %, about 1.8 weight % to about 3.2 weight %, about 2 weight % to about 3 weight %, about 2.2 weight % to about 2.8 weight %, or about 2.4 weight % to about 2.6 weight %, based on a total weight of the personal care composition.

As discussed above, the personal care composition may include a synergistic combination of at least two plant oils. For example, the personal care composition may include a synergistic combination of a first plant oil and a second plant oil. In at least one implementation, a first plant oil and a second plant oil may be present in an effective ratio (i.e., concentration, weight, or volume ratio) or a therapeutically effective ratio (i.e., concentration, weight, or volume ratio) to elicit a response (e.g., biological medical, etc.) of a tissue, system, animal, or human that is being sought. For example, the first plant oil, such as FSO, and the second plant oil, such as HSO, may be present in an effective concentration, weight, or volume ratio or a therapeutically effective concentration, weight, or volume ratio to provide protection on surfaces of the skin against one or more microbes, reduce skin irritation and/or inflammation, and/or provide skin protection benefits against atmospheric or environmental pollution.

In at least one implementation, the concentration, weight, or volume ratio of a first plant oil to a second plant oil may be from about 0.1:1 to about 5:1. For example, the concentration, weight, or volume ratio of the first plant oil to the second plant oil may be from about 0.1:1, about 0.2:1, about 0.3:1, about 0.4:1, about 0.5:1, about 0.6:1, about 0.7:1, about 0.8:1, about 0.9:1, or about 1.0:1 to about 1.1:1, about 1.2:1, about 1.3:1, about 1.4:1, about 1.5:1, about 1.6:1, about 1.7:1, about 1.8:1, about 1.9:1, about 2.0:1, about 2.1:1, about 2.2:1 to about 2.3:1, about 2.4:1, about 2.5:1, about 2.6:1, about 2.7:1, about 2.8:1, about 2.9:1, about 3.0:1, about 3.1:1, about 3.2:1, about 3.3:1, about 3.4:1, about 3.5:1, about 3.6:1, about 3.7:1, about 3.8:1, about 3.9:1, about 4:1, about 4.1:1, about 4.2:1, about 4.3:1, about 4.4:1, about 4.5:1, about 4.6:1, about 4.7:1, about 4.8:1, about 4.9:1, or about 5:1. In another example, the concentration, weight, or volume ratio of the first plant oil to the second plant oil may be from greater than or equal to about 0.1:1 to less than or equal to about 0.2:1, less than or equal to about 0.3:1, less than or equal to about 0.4:1, less than or equal to about 0.5:1, less than or equal to about 0.6:1, less than or equal to about 0.7:1, less than or equal to about 0.8:1, less than or equal to about 0.9:1, less than or equal to about 1.0:1, less than or equal to about 1.1:1, less than or equal to about 1.2:1, less than or equal to about 1.3:1, less than or equal to about 1.4:1, less than or equal to about 1.5:1, less than or equal to about 1.6:1, less than or equal to about 1.7:1, less than or equal to about 1.8:1, less than or equal to about 1.9:1, less than or equal to about 2.0:1, less than or equal to about 2.1:1, less than or equal to about 2.2:1 to less than or equal to about 2.3:1, less than or equal to about 2.4:1, less than or equal to about 2.5:1, less than or equal to about 2.6:1, less than or equal to about 2.7:1, less than or equal to about 2.8:1, less than or equal to about 2.9:1, less than or equal to about 3.0:1, less than or equal to about 3.1:1, less than or equal to about 3.2:1, less than or equal to about 3.3:1, less than or equal to about 3.4:1, less than or equal to about 3.5:1, less than or equal to about 3.6:1, less than or equal to about 3.7:1, less than or equal to about 3.8:1, less than or equal to about 3.9:1, less than or equal to about 4:1, less than or equal to about 4.1:1, less than or equal to about 4.2:1, less than or equal to about 4.3:1, less than or equal to about 4.4:1, less than or equal to about 4.5:1, less than or equal to about 4.6:1, less than or equal to about 4.7:1, less than or equal to about 4.8:1, less than or equal to about 4.9:1, or less than or equal to about 5:1. In yet another example, the concentration, weight, or volume ratio of the first plant oil to the second plant oil may be from greater than or equal to about 0.1:1, greater than or equal to about 0.2:1, greater than or equal to about 0.3:1, greater than or equal to about 0.4:1, greater than or equal to about 0.5:1, greater than or equal to about 0.6:1, greater than or equal to about 0.7:1, greater than or equal to about 0.8:1, greater than or equal to about 0.9:1, greater than or equal to about 1.0:1, greater than or equal to about 1.1:1, greater than or equal to about 1.2:1, greater than or equal to about 1.3:1, greater than or equal to about 1.4:1, greater than or equal to about 1.5:1, greater than or equal to about 1.6:1, greater than or equal to about 1.7:1, greater than or equal to about 1.8:1, greater than or equal to about 1.9:1, greater than or equal to about 2.0:1, greater than or equal to about 2.1:1, greater than or equal to about 2.2:1 to greater than or equal to about 2.3:1, greater than or equal to about 2.4:1, greater than or equal to about 2.5:1, greater than or equal to about 2.6:1, greater than or equal to about 2.7:1, greater than or equal to about 2.8:1, greater than or equal to about 2.9:1, greater than or equal to about 3.0:1, greater than or equal to about 3.1:1, greater than or equal to about 3.2:1, greater than or equal to about 3.3:1, greater than or equal to about 3.4:1, greater than or equal to about 3.5:1, greater than or equal to about 3.6:1, greater than or equal to about 3.7:1, greater than or equal to about 3.8:1, greater than or equal to about 3.9:1, greater than or equal to about 4:1, greater than or equal to about 4.1:1, greater than or equal to about 4.2:1, greater than or equal to about 4.3:1, greater than or equal to about 4.4:1, greater than or equal to about 4.5:1, greater than or equal to about 4.6:1, greater than or equal to about 4.7:1, greater than or equal to about 4.8:1, or greater than or equal to about 4.9:1 to less than or equal to about 5:1. In a particular implementation, the concentration, weight, or volume ratio of the first plant oil to the second plant oil may be from about 0.5:1 to about 4:1, more preferably from about 1:1 to about 2:1. For example, the concentration, weight, or volume ratio of flaxseed oil to hemp seed oil may be from about 0.5:1 to about 4:1, more preferably from about 1:1 to about 2:1.

The total amount of active ingredients in the personal care composition may vary widely. It should be appreciated that the active ingredients of the personal care compositions disclosed herein may include those ingredients capable of or configured to increase the amount of antimicrobial peptides produced by the skin, treat or otherwise reduce skin inflammation and/or irritation, and/or reduce the amount of reactive oxygen species in skin. In at least one example, the active ingredients include, consist essentially of, or consist of the one or more plant oils. For example, the active ingredients of the personal care composition may be any combination of the one or more plant oils disclosed herein. In a preferred implementation, the active ingredients include FSO and HSO. In at least one implementation, the total amount of the active ingredients present in the personal care composition may be from greater than 0 weight % to less than or equal to about 40 weight %, less than or equal to about 30 weight%, less than or equal to about 20 weight %, less than or equal to about 10 weight %, less than or equal to about 9 weight %, less than or equal to about 8 weight %, less than or equal to about 7 weight %, less than or equal to about 6 weight %, less than or equal to about 5 weight %, less than or equal to about 4 weight %, less than or equal to about 3 weight %, less than or equal to about 2 weight %, less than or equal to about 1 weight %, less than or equal to about 0.9 weight %, less than or equal to about 0.8 weight %, less than or equal to about 0.7 weight %, less than or equal to about 0.6 weight %, less than or equal to about 0.5 weight %, less than or equal to about 0.4 weight %, less than or equal to about 0.3 weight %, less than or equal to about 0.2 weight %, less than or equal to about 0.1 weight %, less than or equal to about 0.01 weight %, less than or equal to about 0.001 weight %, less than or equal to about 0.0001 weight %, less than or equal to about 9E-5 weight %, less than or equal to about 8E-5 weight %, less than or equal to about 7E-5 weight %, less than or equal to about 6.875E-5 weight %, less than or equal to about 6.5E-5 weight %, less than or equal to about 6E-5 weight %, less than or equal to about 5.5E-5 weight %, less than or equal to about 5E-5 weight %, or less than or equal to about 4.5E-5 weight %, based on a total weight of the personal care composition. In an exemplary implementation, the active ingredients include flaxseed oil and hemp seed oil in a total weight % of about 0.01 weight % to about 10 weight %, based on a total weight of the personal care composition.

The personal care composition may include the one or more plant oils, mixed with, dissolved in, combined with, or otherwise contacted with the carrier or one or more excipients. In at least one implementation, the carrier may be capable of or configured to store, entrain, or otherwise contain the one or more plant oils, and deliver the one or more plant oils to one or more tissues, such as skin. It should be appreciated that the components or contents of the carrier and the respective amount of each of the components of the carrier may be at least partially determined by the type or use of the personal care product or the composition thereof. Illustrative personal care products or compositions thereof may include, but are not limited to, cleansers, leave-on skin lotions or creams, emulsion, shampoos, conditioners, shower gels, antiperspirants, deodorants, depilatories, lipsticks, foundations, mascara, sunless tanners, sunscreen lotions, body washes, soaps, including bar soaps and liquid soaps (e.g., liquid hand soaps), face washes, moisturizers, serums, spot treatments, cosmetics, or the like. In a preferred implementation, the personal care product or the composition thereof that includes the one or more plant oils are solid cleansing compositions, such as bar soaps.

In at least one implementation, the personal care product or the composition thereof may be a skin care product. Illustrative skin care product may be or include, but are not limited to, a lotion, a cosmetic, a sunscreen, or the like. The carrier of the skin care product may include, but is not limited to, any one or more of surfactants, conditioning agents, moisturizers, sunscreens, UV absorbers, antioxidants, enzymes and/or other proteins, vitamins, antibacterial agents, odor reducing agents, steroids, anti-inflammatory agents, naturally and/or non-naturally occurring humectants, skin lipid fluidizers, occlusive agents, amino acids, physical and/or chemical exfoliants, skin whiteners, anti-aging, antiperspirant actives, or the like, or any combination thereof.

In at least one implementation, the personal care product or the composition thereof may be a personal hand and/or body cleansing composition or a personal hand and/or body conditioning composition. Illustrative personal hand and/or body cleansing or conditioning compositions may include, but are not limited to, liquid soaps, bar soaps, body washes, shower gels, lotions, and the like. In a preferred implementation, the personal hand and/or body cleansing or conditioning composition is a solid personal hand and/or solid body cleansing or conditioning composition, such as bar soap. The carrier for the personal hand and/or body cleansing composition or the personal hand and/or body conditioning composition may include, but is not limited to, any one or more of fragrances, essential oils, emulsifying agents, thickening agents, colorants, surfactants, natural actives, therapeutic actives, stain prevention actives, antimicrobial agents, vitamins, natural extracts, amino acids, enzymes and/or other proteins, abrasives, odor control agents, conditioning agents, moisturizers, humectants, occlusive agents, skin lipid fluidizers, lipophilic actives, hydrophilic materials, pearlizers, opacifying agents, sodium soaps, titanium dioxide, fragrances, or the like, or any mixture or combination thereof, in addition to any one or more of the other carrier components as discussed above.

The carrier may be hydrophilic or hydrophobic. The carrier may be anhydrous. The carrier may be a liquid or a solid at room temperature. The carrier may have a viscosity of from about 2,000 centipoise (cP) to about 100,000 cP. For example, the carrier for a shower gel may have a viscosity of from about 2,000 cP to about 16,000 cP. In another example, the carrier for a lotion may have a viscosity of from about 10,000 cP to about 100,000 cP. Accordingly, it should be appreciated that the viscosity of the carrier may vary and may at least partially depend on the type of personal care composition. In an exemplary implementation, the carrier is a solid at room temperature.

Unless otherwise specifically identified, the ingredients for use in the compositions and formulations of the compositions disclosed herein are preferably cosmetically acceptable ingredients. As used herein, the expression “cosmetically acceptable” may refer to a component or ingredient that is suitable for use in a formulation for topical application to human skin. A cosmetically acceptable excipient, may refer to an excipient that is suitable for external application in the amounts and concentrations contemplated in the formulations of the compositions disclosed herein, and includes for example, excipients which are “Generally Recognized as Safe” (GRAS) by the United States Food and Drug Administration (FDA).

### METHODS

The present disclosure may provide methods for preparing a personal care product or a personal care composition thereof. The method may include mixing, stirring, combining, or otherwise contacting a carrier and one or more plant oils with one another. For example, the method may include contacting the carrier, flaxseed oil, and hempseed oil with one another. In at least one example, the carrier may be a solid cleansing composition, such as a bar soap.

The present disclosure may also provide methods for treating microbes on skin and/or providing protection on surfaces of the skin from microbes. The method may include increasing production of AMPs in and/or on the skin by contacting any one or more of the personal care compositions disclosed herein or the plant oils thereof with the skin. The method may also include reducing the amount of microbes on the skin by increasing the production of AMPs in the skin. The method may also include increasing production of AMP biomarker LL-37 and/or cathelicidins in and/or on the skin. The method for treating microbes on skin may also include enhancing or increasing the innate immune response by treating the skin with any one or more of the personal care compositions disclosed herein. The one or more microbes may be or include, but are not limited to, Gram negative bacteria, Gram positive bacteria, any strains that are resistant to conventional antibiotics, mycobacteria, enveloped viruses, fungi, transformed and/or cancerous cells, other microbes, or the like, or any combination thereof.

The present disclosure may further provide methods for treating or reducing one or more skin conditions, such as skin irritation or skin inflammation. Illustrative irritation or inflammatory conditions may be or include, but are not limited to, redness, itchiness, pruritus, pyoderma, dermal rashes, chaffing, or the like, or combinations thereof. The method for treating or reducing one or more skin conditions may include decreasing the amount of IL-1α in and/or on the skin by contacting an effective amount of any one or more of the personal care compositions disclosed herein or the plant oils thereof with the skin. The method may also include diagnosing or determining the presence of skin irritation and/or skin inflammation. The method for diagnosing or determining the presence of skin irritation and/or skin inflammation may include measuring an elevated amount of IL-1α relative to a population baseline value or a previous individual baseline value. It should be appreciated that the population baseline value may be the value from a population that does not have skin irritation or skin inflammation. The elevated amount of IL-1α may be at least 1% greater, at least 2% greater, at least 5% greater, at least 8% greater, at least 10% greater, at least 12% greater, at least 15% greater, at least 20% greater, at least 25% greater, at least 30% greater, at least 40% greater, at least 50% greater, at least 60% greater, at least 70% greater, at least 80% greater, at least 90% greater, at least 100% greater, at least 150% greater, at least 200% greater, at least 250% greater, at least 300% greater, at least 400% greater, or at least 500% greater than the population or previous individual baseline value.

The present disclosure may further provide methods for treating, decreasing, reducing, or preventing damage to the skin and/or providing skin protection benefits against atmospheric or environmental pollution by reducing reactive oxygen species in and on the skin. The method may include contacting an effective amount of any one or more of the personal care compositions disclosed herein or the plant oils thereof with the skin.

The present disclosure may also provide a personal care composition including a carrier and one or more plant oils for use in treating microbes on skin.

The present disclosure may also provide a personal care composition including a carrier and one or more plant oils for use in treating one or more skin conditions, such as skin irritation and/or skin inflammation.

The present disclosure may also provide a personal care composition including a carrier and one or more plant oils for use in treating, decreasing, reducing, or preventing damage to the skin and/or providing skin protection benefits against atmospheric or environmental pollution by reducing reactive oxygen species in and on the skin.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

An in vitro study was conducted on keratinocyte cell skin models to determine the efficacy of varying concentrations of hemp seed oil (HSO) and/or flaxseed oil (F SO) for the production of antimicrobial peptides (AMPs). The production of AMPs was determined by measuring the presence of AMP biomarkers, namely, LL-37. It should be appreciated that AMPs, also called host defense peptides, are part of the innate immune response. AMPs have demonstrated efficacy as therapeutic agents, and have further demonstrated the ability to treat, reduce, or eliminate Gram-negative and Gram-positive bacteria, including those resistant to conventional antibiotics, mycobacteria, enveloped viruses, fungi, and transformed or cancerous cells. According to the foregoing, it should be appreciated that AMPs provide protection for epithelial surfaces or skin from environmental microbes.

To prepare the varying concentrations or dilutions of HSO and/or FSO, stock solutions of the HSO and the FSO were prepared. 100% FSO was combined with 100% dimethyl sulfoxide (DMSO) in a ratio of about 1:99 to thereby prepare a 1% solution of FSO in DMSO. Similarly, HSO was combined with 100% DMSO in a ratio of about 1:99 to thereby prepare a 1% solution of HSO in DMSO. Each of the 1% solutions of FSO and HSO was then combined about 900 μL of phosphate-buffered saline (PBS) to prepare respective 0.1 wt% solutions of FSO and HSO. A stock 10% DMSO solution was prepared by combining 100 μL of DMSO with 900 μL of PBS.

The stock solutions were utilized to prepare six separate samples (1)-(6) having varying amounts of FSO and/or HSO. The amounts or concentrations of each of the samples (1)-(6) are summarized in Table 1. As indicated in Table 1, sample (1) only included media that included nutrient for tissue growth, sample (2) included about 0.001% FSO, sample (3) included about 0.001% HSO, sample (4) included about 0.002% HSO, sample (5) included about 0.001% FSO and about 0.001% HSO, and sample (6) included about 0.001% FSO and about 0.002% HSO.

TABLE 1
Composition of Samples (1)-(6)

| | 1 | 2 | 3 | 4 | 5 | 6 |
|---|---|---|---|---|---|---|
| Ingredient | mL | mL | mL | mL | mL | mL |
| FSO (0.1%) | 0 | 0.1 | 0 | 0 | 0.1 | 0.1 |
| HSO (0.1%) | 0 | 0 | 0.1 | 0.2 | 0.1 | 0.2 |
| Media | 10.0 | 9.9 | 9.9 | 9.8 | 9.8 | 9.7 |

Keratinocyte cell skin models were utilized as the models in the in vitro study, and LL-37 production was monitored with an ELISA Kit. To conduct the in vitro study, Keratinocyte cell were grown in a 6-well plate to reach the confluency of about >60%. About 2 mL of each of the samples (ingredient diluted in the media) (1)-(6) was directly applied to respective wells of a well plate and incubated for 24 hours. After incubation, the cells from each of the wells were collected and used as is. The production of AMP biomarker LL-37, as measured by the ELISA kit, from respective samples (1)-(6) is summarized in Table 2. All measurements were done in triplicate, averaged, and normalized to the total protein in each of the keratinocyte cell skin models.

TABLE 2
Amount of LL-37 Measured from Skin
Models Treated with Samples (1)-(6)

| # | Sample | Amount of LL-37 | Change from Control |
|---|---|---|---|
| 1 | Control; Media Only | 17.9 | 0 |
| 2 | 0.001% FSO | 20.5 | 2.6 |
| 3 | 0.001% HSO | 21.1 | 3.2 |
| 4 | 0.002% HSO | 19.8 | 1.9 |
| 5 | 0.001% FSO + | 25.2 | 7.3 |
| | 0.001% HSO | | |
| 6 | 0.001% FSO + | 22.2 | 4.3 |
| | 0.002% HSO | | |

Based on the changes relative to control for samples (2) and (4), which included 0.001% FSO and 0.002% HSO, respectively, it was expected that the amount of LL-37 in sample (6) would be an increase of about 4.5. An increase of about 4.3 was observed and substantially similar to the expected amount of 4.5. However, when decreasing the amount or ratio of HSO to FSO (or increasing the amount or ratio of FSO to HSO), as in sample (5), it was surprisingly and unexpectedly discovered that the amount of LL-37 measured increased significantly. Particularly, based on the increase of about 2.6 and about 3.2 observed in samples (2) and (3), respectively, an increase of about 5.8 was expected. However, when the amount or ratio of HSO to FSO was decreased in sample (5) the amount of LL-37 measured was actually about 7.3, which was significantly greater than the 5.8 expected. It should be appreciated that the increase in biomarker LL-37 indicates an increased production of antimicrobial peptide (AMP), which was surprising and unexpected. The results suggest that providing a ratio of FSO to HSO of less than about 1:2 and/or less than or equal to about 1:1 surprisingly and unexpectedly increases the production of AMPs in skin.

### Example 2

An in vitro study was conducted on keratinocyte cell skin models to observe the production of Interleukin 1 alpha (IL-1α) in the presence of varying concentrations of HSO and/or FSO. It should be appreciated that IL-1α, which is also known as hematopoietin 1, is a cytokine of the interleukin 1 family that is encoded by the IL1α gene. It should further be appreciated that interleukin 1 is responsible for the production of inflammation and irritation, and is widely accepted as a biomarker to evaluate skin irritation.

To evaluate the IL-1α production, the keratinocyte cell skin models were grown to at least 60% confluency and placed in respective wells of a well plate. Stock solutions of FSO and HSO were prepared similar to Example 1. Particularly, 100% FSO was combined with 100% dimethyl sulfoxide (DMSO) in a ratio of about 1:99 to thereby prepare a 1% solution of FSO in DMSO. Similarly, HSO was combined with 100% DMSO in a ratio of about 1:99 to thereby prepare a 1% solution of HSO in DMSO. A vehicle stock solution including 0.2% DMSO was prepared by combining 200 μL of 10% DMSO with 9.8 mL media for growing the keratinocyte cells.

The stock solutions were utilized to prepare eight samples (7)-(14) having varying amounts of FSO and/or HSO. The amounts or concentrations of each of the samples (7)-(14) are summarized in Table 3. A brief description of each of the samples (7)-(14) is summarized in Table 4.

TABLE 3
Composition of Samples (7)-(14)

| | 7 | 8 | 9 | 10 | 11 | 12 | 13 | 14 |
|---|---|---|---|---|---|---|---|---|
| Ingredient | mL | mL | mL | mL | mL | mL | mL | mL |
| FSO (0.1%) | 0 | 0.1 | 0 | 0 | 0 | 0.1 | 0.1 | 0.1 |
| HSO (0.1%) | 0 | 0 | 0.1 | 0.05 | 0.025 | 0.1 | 0.05 | 0.025 |
| Media | 9.7 | 9.8 | 9.8 | 9.85 | 9.875 | 9.7 | 9.75 | 9.775 |
| 30% Peroxide | 0.1 | 0.1 | 0.1 | 0.1 | 0.1 | 0.1 | 0.1 | 0.1 |
| (H₂O₂) | | | | | | | | |
| 10% DMSO | 0.2 | 0 | 0 | 0 | 0 | 0 | 0 | 0 |

TABLE 4
Brief Description of Samples (7)-(18)

| Sample | Brief Description |
|---|---|
| 7 | Negative Control |
| 8 | 0.001% FSO |
| 9 | 0.001% HSO |
| 10 | 0.0005% HSO |
| 11 | 0.00025% HSO |
| 12 | 0.001% FSO + |
| | 0.001% HSO |
| 13 | 0.001% FSO + |
| | 0.0005% HSO |
| 14 | 0.001% FSO + |
| | 0.00025% HSO |

Each of the samples (7)-(14) were used directly in IL-1α assays with IL-1α ELISA kits. All measurements were done in triplicate and averaged. The results of the IL-1α assay are summarized in Table 5.

TABLE 5
Amount of IL-1α Measured in Samples (7)-(14)

| # | Sample | Amount of IL-1α | Change from Control |
|---|---|---|---|
| 7 | Negative Control | 13.4 | — |
| 8 | 0.001% FSO | 13.8 | 0.4 |
| 9 | 0.001% HSO | 13.0 | −0.4 |
| 10 | 0.0005% HSO | 11.1 | −2.3 |
| 11 | 0.00025% HSO | 11.6 | −1.8 |
| 12 | 0.001% FSO + | 8.9 | −4.5 |
| | 0.001% HSO | | |
| 13 | 0.001% FSO + | 8.6 | −5.2 |
| | 0.0005% HSO | | |
| 14 | 0.001% FSO + | 12.3 | −1.1 |
| | 0.00025% HSO | | |

As illustrated in Table 5, the combination of FSO and HSO surprisingly, unexpectedly, and significantly decreased the amount of IL-1α measured. For example, based on the amount of IL-1α measured in samples (7)-(11), the expected amounts of IL-1α measured in samples (12) and (13) were about 0 and about −2.7, respectively. As indicated in Table 5, however, the amount of IL-1α measured in samples (12) and (13) was actually about −4.5 and about −5.2, respectively. Regarding sample (14), no synergy was observed. Particularly, the amount of IL-1α expected was about −1.4, and about −1.1 was measured. The demonstrated synergistic combination of FSO and HSO at ratios of about 1:1 and about 2:1, and the lack of synergy at a ratio of about 4:1 suggests that providing FSO and HSO at ratios of less than about 4:1, or less than 2:1, surprisingly and unexpectedly decreases the amount of IL-1α measured, thereby reducing skin irritation.

### Example 3

An in vitro study similar to Example 2 was conducted on keratinocyte cell skin models to observe the production of Interleukin 1 alpha (IL-1α) in the presence of varying concentrations of hemp HSO and/or FSO. The stock solutions prepared in Example 2 were utilized in the study. The stock solutions were utilized to prepare eight samples (15)-(22) having varying amounts of FSO and/or HSO. The amounts or concentrations of each of the samples (15)-(22) are summarized in Table 6. A brief description of each of the samples (15)-(22) is summarized in Table 7.

TABLE 6
Composition of Samples (15)-(22)

| | 15 | 16 | 17 | 18 | 19 | 20 | 21 | 22 |
|---|---|---|---|---|---|---|---|---|
| Ingredient | mL | mL | mL | mL | mL | mL | mL | mL |
| FSO (0.1%) | 0 | 0.05 | 0 | 0 | 0 | 0.05 | 0.05 | 0.05 |
| HSO (0.1%) | 0 | 0 | 0.1 | 0.05 | 0.025 | 0.1 | 0.05 | 0.025 |
| Media | 9.7 | 9.95 | 9.9 | 9.95 | 9.975 | 9.85 | 9.9 | 9.925 |
| 30% Peroxide | 0.1 | 0 | 0 | 0 | 0 | 0 | 0 | 0 |
| (H₂O₂) | | | | | | | | |
| 10% DMSO | 0.2 | 0 | 0 | 0 | 0 | 0 | 0 | 0 |

TABLE 7
Brief Description of Samples (15)-(22)

| Sample | Brief Description |
|---|---|
| 15 | Negative Control |
| 16 | 0.0005% FSO |
| 17 | 0.001% HSO |
| 18 | 0.0005% HSO |
| 19 | 0.00025% HSO |
| 20 | 0.0005% FSO + |
| | 0.001% HSO |
| 21 | 0.0005% FSO + |
| | 0.0005% HSO |
| 22 | 0.0005% FSO + |
| | 0.00025% HSO |

Each of the samples (15)-(22) were used directly in IL-1α assays with IL-1α ELISA kits. All measurements were done in triplicate and averaged. The results of the IL-1α assay are summarized in Table 8. It is noted that samples (18) and (19) included 0.0005% HSO and 0.00025% HSO, similar to samples (10) and (11) of Example 2, and the amount of IL-1α measured in Example 2 and Example 3 were different. It should be appreciated by one skilled in the art that the variation in the amount of IL-1α measured in Examples 2 and 3 may be attributed to the variation in the growth of the keratinocyte cell skin models in each study or set of cells. For example, variables such as methods and amount of cells plated may vary the concentration from one study/set to another. As such, samples including 0.0005% HSO and 0.00025% HSO were performed again in Example 3 to provide comparable results.

TABLE 8
Amount of IL-1α Measured in Samples (15)-(22)

| # | Sample | Amount of IL-1α | Change from Control |
|---|---|---|---|
| 15 | Negative Control | 39.8 | — |
| 16 | 0.0005% FSO | 33.9 | −5.9 |
| 17 | 0.001% HSO | 34.5 | −5.4 |
| 18 | 0.0005% HSO | 47 | 8.2 |
| 19 | 0.00025% HSO | 46.1 | 6.3 |
| 20 | 0.0005% FSO + | 41.9 | 2.0 |
| | 0.001% HSO | | |
| 21 | 0.0005% FSO + | 37.9 | −2.0 |
| | 0.0005% HSO | | |
| 22 | 0.0005% FSO + | 37.5 | −2.3 |
| | 0.00025% HSO | | |

As illustrated in Table 8, the combination of FSO and HSO surprisingly, unexpectedly, and significantly decreased the amount of IL-1α measured. For example, based on the amount of IL-1α measured in samples (15)-(19), the expected amounts of IL-1α measured in samples (21) and (22) were about 2.3 and 0.4, respectively. As indicated in Table 8, however, the amount of IL-1α measured in samples (21) and (22) was actually about −2.0 and about −2.3, respectively. Regarding sample (20), a decrease of about −11.3 was expected, but an increase of about 2.0 was observed. While the combination of FSO to HSO in sample (20) was synergistic, the combination demonstrated an increase in the IL-1α measured. The foregoing results of Example 3 suggest that providing FSO and HSO in a ratio of about 1:1 and about 2:1 surprisingly and unexpectedly decreased the amount of IL-1α measured significantly. The results of Example 3, particularly, sample (20) suggests that providing FSO and HSO at a ratio of greater than about 0.5:1 surprisingly and unexpectedly decreased the amount of IL-1α measured in the cells.

In view of the results disclosed in Examples 2 and 3, it should be appreciated that a surprising and unexpected decrease in IL-la is observed when a ratio of FSO to HSO is provided in an amount greater than about 0.5:1 and less than about 4:1. The synergistic results are further enhanced when a ratio of FSO to HSO is provided in an amount of from about 1:1 to about 2:1.

### Example 4

An in vitro study was conducted to analyze the skin protecting benefits of varying concentrations of HSO and/or FSO. Particularly, the ability of HSO and/or FSO in preventing the generation of or reducing the presence of reactive oxygen species (ROS) was evaluated. Skin is often exposed to various environmental stressors, including air pollution. One of the most harmful components of air pollution is particulate matter (PM), which causes skin damage through PM-induced oxidative stress. In PM-induced oxidative stress, PM deposited on skin react with the skin to generate ROS in and on the skin that can lead to oxidative cell damage and cell death. Accordingly, the present in vitro study was conducted to analyze the efficacy of varying concentrations of HSO and/or FSO in reducing and/or preventing ROS in and on the skin.

To conduct the study, neonatal human epidermal keratinocytes (HEKn) were grown in standard well plates having glass bottoms coated with collagen I. The HEKn were cultured in EpiLife™ media supplemented with human keratinocyte growth supplements (HKGS), both of which are commercially available from ThermoFisher Scientific of Waltham, Mass. Stock solutions of FSO, HSO, and urban dust (UD) were prepared for the treatment of the keratinocytes. It should be appreciated that the UD is representative of particulate matter (PM) pollution.

The presence or amount of intracellular reactive oxygen species (ROS) was measured using fluorescence microscopy. Specifically, the keratinocytes were exposed to an oxidative stress detection reagent for about one hour in the absence (i.e., control) or presence of FSO, HSO, and/or UD. After exposure to the oxidative stress detection reagent and the FSO, HSO, and/or UD, the keratinocytes were washed twice with a wash buffer and imaged via fluorescence in the wash buffer. Live adherent cells were imaged using GFP excitation/emission filters, and the ROS levels were quantified from raw images using ImageJ software. The measured ROS levels in each of the samples are summarized in Table 9.

TABLE 9
ROS Levels (%)Measured in Samples (23)-(32)

| | | | | | Standard |
|---|---|---|---|---|---|
| | | | | ROS Levels (%) | Error |
| | UD | HSO | FSO | Relative to | of Mean |
| Sample | (μg/mL) | (%) | (%) | No Treatment | (SEM) |
| 23 | 200 | 0 | 0 | 0 | 6.35 |
| Control | | | | | |
| 24 | 200 | 0 | 0 | 189.54 | 4.48 |
| 25 | 200 | 1.25E−4 | 0 | −5.92 | 3.11 |
| 26 | 200 | 6.25E−5 | 0 | −0.26 | 1.17 |
| 27 | 200 | 0 | 1.25E−4 | −10.16 | 2.71 |
| 28 | 200 | 0 | 6.25E−5 | −6.9 | 1.04 |
| 29 | 200 | 1.25E−4 | 1.25E−4 | −10.39 | 1.86 |
| 30 | 200 | 1.25E−4 | 6.25E−5 | −11.82 | 1.89 |
| 31 | 200 | 6.25E−5 | 1.25E−4 | −12.25 | 1.68 |
| 32 | 200 | 6.25E−5 | 6.25E−5 | −12.85 | 1.93 |

As illustrated in Table 9, the combination of FSO and HSO surprisingly, unexpectedly, and significantly reduced the amount of ROS levels measured. Particularly, based on the ROS levels measured in samples (26) and (28), a decrease of about 7% was expected when the FSO and the HSO were combined in a ratio of about 1:1, as in sample (32). However, when the FSO and the HSO were combined in a ratio of about 1:1, a statistically significant decrease of about −13% (P=0.00007) was observed in sample (32).

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof.

1. A personal care composition, comprising a carrier and at least two plant oils, wherein the at least two plant oils are present in an effective amount to increase the amount of antimicrobial peptides produced by skin, reduce inflammation of skin, reduce irritation of skin, reduce reactive oxygen species in skin, or a combination thereof.

2. The personal care composition of claim 1, wherein the at least two plant oils comprise flaxseed oil and hemp seed oil.

3. The personal care composition of claim 2, wherein the flaxseed oil and the hemp seed oil are present in a ratio of about 0.5:1 to about 4:1, more preferably about 1:1 to about 2:1.

4. The personal care composition of claim 1, wherein the at least two plant oils are present in an amount of from greater than 0 weight % to about 40 weight %, based on a total weight of the personal care composition.

5. The personal care composition of claim 1, wherein at least one of the plant oils is a manufactured oil.

6. The personal care composition of claim 1, wherein at least one of the plant oils is a natural oil.

7. The personal care composition of claim 1, wherein the personal care composition consists essentially of the carrier and the at least two plant oils.

8. The personal care composition of claim 1, wherein the personal care composition consists of the carrier and the at least two plant oils.

9. The personal care composition of claim 1, wherein the carrier is a solid carrier.

10. The personal care composition of claim 1, wherein the carrier is a liquid carrier.

11. A method for preparing the personal care composition of claim 1, the method comprising contacting the carrier and the at least two plant oils with one another.

12. A method for treating, preventing, or reducing microbes on skin, the method comprising contacting the skin with the personal care composition of claim 1, optionally, the method further comprising increasing production of antimicrobial peptides in or on the skin.

13. The method of claim 12, wherein contacting the skin increases production of antimicrobial peptide biomarker LL-37 in or on the skin.

14. The method of claim 12, wherein contacting the skin increases an innate immune response.

15. A method for treating or reducing skin irritation or skin inflammation, the method comprising contacting the skin with the personal care composition of claim 1, optionally, the method further comprising decreasing the amount of IL-1α in or on the skin.

16. A method for treating or reducing reactive oxygen species in or on skin, the method comprising contacting the skin with the personal care composition of claim 1.

17. The personal care composition of claim 1 for use in treating, preventing, or reducing microbes on skin, optionally, wherein the personal care composition increases production of antimicrobial peptides in or on the skin.

18. The personal care composition of claim 1 for use in increasing an innate immune response on skin.

19. The personal care composition of claim 1 for use in treating or reducing skin irritation or skin inflammation.

20. The personal care composition of claim 1 for use in treating or reducing reactive oxygen species in or on skin.
